(19) United States
(12) Patent Application Publication
(10) Pub. No.: US 2017/0246166 A1
(43) Pub. Date: Aug. 31, 2017

(54) 1,2-BENZOTHIAZOLE COMPOUNDS FOR THE TREATMENT OF KIDNEY DISORDERS

(71) Applicant: Eli Lilly and Company, Indianapolis, IN (US)

(72) Inventors: Michael James Genin, I, Danville, IN (US); William Glen Holloway, Brownsburg, IN (US); Mark David Rekhter, Carmel, IN (US)

(21) Appl. No.: 15/513,161

(22) PCT Filed: Nov. 13, 2015
(86) PCT No.: PCT/US2015/060635

§ 371 Date: Mar. 22, 2017

Related U.S. Application Data
(60) Provisional application No. 62/082,655, filed on Nov. 21, 2014.

(51) Int. Cl. A61K 31/496; C07D 417/06

(52) U.S. Cl. CPC A61K 31/496; C07D 417/06

(57) ABSTRACT

The present invention provides 1,2-benzothiazole compounds particularly useful in the treatment of diabetic nephropathy and other chronic kidney disorders and other diabetic complications.

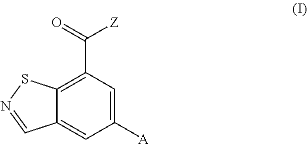

Renal or kidney disorders in man and animals involve an alteration in the normal physiology and function of the kidney. Renal disorders can result from a wide range of acute and chronic conditions and events, including physical, chemical, or biological injury, insult or trauma, disease, and various inflammatory and autoimmune diseases. Kidney disorders can lead to reduced kidney function, seriously compromising quality and duration of life. Regardless of the initial insult or cause, kidney disorders are characterized by progressive destruction of the renal parenchyma and the loss of functional nephrons. This progression often leads to chronic kidney disease (CKD) and end-stage renal disease and failure (ESRD/ESRF).

CKD is characterized by the progressive loss of kidney function. Increased albuminuria and gradual, progressive loss of renal function are primary manifestations in CKD. Decreased renal function results in increased blood creatinine and blood urea nitrogen (BUN). CKD patients experience over time an increase in albuminuria, proteinuria, serum creatinine, and renal histopathological lesions.

In humans, CKD has been, and continues to be, a considerable social and economic problem in all industrialized countries. In the USA, 102,567 patients began dialysis in 2003 (341 patients/year per million), and similar rates were found in developing countries and in particular ethnic groups (2006, USRDS Am J Kidney Dis 47:1-286; Meguid El Nahas, A., and Bello, A. K. 2005. Chronic kidney disease: the global challenge. Lancet 365:331-340.). However, these numbers are a small fraction of the millions of patients who are thought to have some degree of renal impairment. In the United States, the prevalence of chronically reduced kidney function is estimated to be around 10% of adults (http://kidney.niddk.nih.gov/kudiseases/pubs/kustats/index.htm, pages 1-4). Worsening CKD evolves into ESRD for many patients, requiring either dialysis or kidney transplant. Glomerular filtration rate (GFR) is used to classify the severity of CKD for patients, with lower GFR corresponding to more severe CKD. Reducing the rate at which GFR declines in patients is expected to delay or prevent the development of ESRD. Angiotensin converting enzyme (ACE) inhibitors e.g., benazepril, captopril, enalapril, fosinopril, lisinopril, moexipril, perindopril, quinapril, ramipril, and trandolapril; or angiotensin II receptor antagonists or blockers (ARBs) e.g., candesartan, eprosartan, irbesartan, telmisartan, valsartan, losartan, and olmesartan; or combinations thereof, are used as current standard of care to slow the progression of CKD to ERSD, but these have been shown inadequate to stop the ultimate progression to dialysis.

The prevalence of renal disorders is also high in cats, whereas chronic renal failure is considered the most important one. The prevalence of feline CKD has been reported to reach up to 20%, with 53% of those cats being older than 7 years (Lefebre, Toutain 2004, J. Vet. Pharm. Therap. 27, 265-281; Wolf, North. Am. Vet Congress 2006). Survival in cats with mild to moderate azotemia and extrarenal clinical signs (International Renal Interest Society (IRIS) stages 2 and 3) ranges from 1 to 3 years. Current therapy aims to delay the progression of the disease in cats by improving renal function. This includes dietary protein restriction, modification of dietary lipid intake, phosphate restriction, and treatment with ACE inhibitors (P. J. Barber (2004) The Kidney, in: Chandler E A, Gaskell C J, Gaskell R M, (eds.) Feline Medicine and Therapeutics, 3rd edition, Blackwell Publishing, Oxford, UK).

There remains a need in the art to provide alternative therapies for treating kidney disorders in man and animals. Particularly acute needs are alternative therapies for treating human and feline CKD.

A particularly high risk group for CKD include those with diabetes. Diabetic nephropathy is chronic kidney disease or damage that results as a complication of diabetes and is the leading cause of ERSD. Thus, diabetic nephropathy is both a subset of chronic kidney disease and a complication of diabetes. The overall risk of developing diabetic nephropathy varies between about 10% of type II diabetics (diabetes of late onset) to about 30% of type I diabetics (diabetes of early onset). It is believed that hyperglycemia (uncontrolled high blood sugar) leads to the development of kidney damage, especially when high blood pressure is also present.

Multiple biochemical pathways have been proposed to explain the adverse effects of hyperglycemia including activation of the diacylglycerol (DAG)-protein kinase C (PKC) pathway. PKC is a family of serine/threonine kinases consisting of 12 isoforms: conventional PKCs (α, β1, β2, and γ) that bind both Ca²⁺ and DAG, novel PKCs (δ, ε, η, θ, and μ) that bind DAG, but not Ca²⁺, and atypical PKCs (ζ, λ, and ι) that bind neither. The activation of conventional and novel PKC isoforms requires the correct phosphorylation of the isoforms and the presence of cofactors such as Ca²⁺ and DAG. When properly phosphorylated, rapid or chronic increases in Ca²⁺ or DAG will induce its translocation to the membranous compartments of the cells to elicit biological actions. Rapid and short-term increases of DAG and Ca²⁺ levels are usually induced by cytokines via the activation of phospholipase C. Chronic activation of PKCs requires sustained elevations of DAG, which involves the activation of phospholipase D/C or the de novo synthesis of DAG. PKC activation directly increases the permeability of albumin and other macromolecules through barriers formed by endothelial cells. In the hyperglycemic and diabetic states, all of these pathways probably contribute to the activation of the DAG-PKC cascade.

PKC inhibitors are already known in the art for the treatment of certain diabetic complications; see for example, U.S. Pat. No. 5,552,386 and U.S. Pat. No. 5,710,145.

Currently, there is no cure for diabetic nephropathy. Diabetic nephropathy, as with CKD, is initially treated with medicines that lower blood pressure, such as ACE inhibitors, ARBs, or combinations thereof. These classes of compounds also appear to exhibit anti-inflammatory effects. Unfortunately, such treatments only slow disease progression and are not successful in halting the progression or repairing damage done to the kidneys. Treatments eventually become more aggressive (dialysis and/or kidney transplantation) as the kidneys deteriorate towards failure.

Therefore, there exists a need for alternative compounds for diabetic nephropathy. Preferably such compounds would be more efficacious and could optionally be combined with an ACE inhibitor, an ARB, or a combination thereof. Preferably such compounds would not inhibit Akt, a signaling molecule in the insulin signaling pathway, but would inhibit the activation of conventional and novel PKC isoforms that could provide treatment for diabetic complications such as atherosclerosis, cardiomyopathy, retinopathy, nephropathy, and neuropathy.

The present invention provides a compound of the formula:

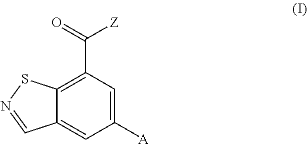

or a salt thereof, wherein:

A is

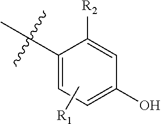

R₁ and R₂ are each independently hydrogen, fluoro, chloro, methyl, or cyano, wherein at least one of R₁ or R₂ is not hydrogen;

Z is

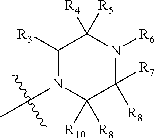

R₃, R₆, R₉, and R₁₀ are each independently hydrogen or methyl;

R₄ and R₅ are each independently hydrogen or methyl, or R₄ and R₅ taken together with the carbon to which they are attached form cyclopropyl; and

R₇ and R₈ are each independently hydrogen or methyl, or R₇ and R₈ taken together with the carbon to which they are attached form cyclopropyl; wherein at least one of R₃, R₄, R₅, R₇, R₈, R₉, or R₁₀ is not hydrogen. The compound of formula I may have clinical use in the treatment of kidney disorders, including chronic kidney disease, and more particularly diabetic nephropathy. Further, a compound of formula I may have clinical use in the treatment of diabetic complications other than or in addition to diabetic nephropathy, such as atherosclerosis, cardiomyopathy, retinopathy, and neuropathy.

In an embodiment of the invention, R₁ and R₂ are each independently hydrogen, fluoro, chloro, or methyl. In an embodiment of the invention, R₁ and R₂ are each independently fluoro, chloro, methyl, or cyano. In an embodiment of the invention, R₁ and R₂ are each independently fluoro, chloro, or methyl. In an embodiment of the invention, R₁ and R₂ are each independently fluoro or chloro. In an embodiment of the invention, R₁ and R₂ are each independently fluoro or methyl. In an embodiment of the invention, R₁ and R₂ are each independently chloro or methyl. In an embodiment of the invention, R₁ and R₂ are each fluoro. In an embodiment of the invention, R₁ is hydrogen. In an embodiment of the invention, R₆ is hydrogen. In an embodiment of the invention, R₆ is methyl. In an embodiment of the invention, R₄ and R₅ are each independently hydrogen or methyl. In an embodiment of the invention, R₇ and R₈ are each independently hydrogen or methyl. In an embodiment of the invention, R₃ is hydrogen. In an embodiment of the invention, R₃ is methyl. In an embodiment of the invention, R₄ is hydrogen. In an embodiment of the invention, R₄ is methyl. In an embodiment of the invention, R₅ is hydrogen. In an embodiment of the invention, R₅ is methyl. In an embodiment of the invention, R₇ is hydrogen. In an embodiment of the invention, R₇ is methyl. In an embodiment of the invention, R₈ is hydrogen. In an embodiment of the invention, R₈ is methyl. In an embodiment of the invention, R₉ is hydrogen. In an embodiment of the invention, R₉ is methyl. In an embodiment of the invention, R₁₀ is hydrogen. In an embodiment of the invention, R₁₀ is methyl. In an embodiment of the invention, if R₄ and R₅ are taken together with the carbon to which they are attached to form cyclopropyl, then R₇ and R₈ are not taken together with the carbon to which they are attached to form cyclopropyl. In an embodiment of the invention, if R₇ and R₈ are taken together with the carbon to which they are attached to form cyclopropyl, then R₄ and R₅ are not taken together with the carbon to which they are attached to form cyclopropyl.

In a preferred embodiment of the invention, A is

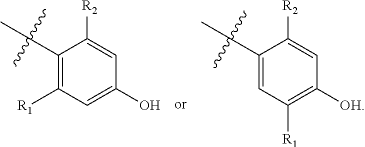

In a preferred embodiment of the invention, R₂ is fluoro. In a preferred embodiment of the invention, A is

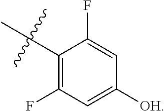 [SMILES: *c1c(F)cc(O)cc1F]

In a preferred embodiment of the invention, Z is

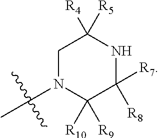

In a preferred embodiment of the invention, Z is

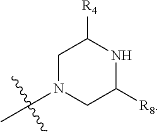

In a preferred embodiment of the invention, Z is

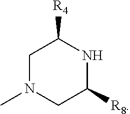

The present invention also provides a method of treating a kidney disorder (such as CKD or diabetic nephropathy) and/or a diabetic complication in a patient comprising administering to a patient in need of such treatment an effective amount of a compound or salt thereof of the present invention. The present invention also provides the above methods further including administering in simultaneous, separate, or sequential combination an additional active ingredient, such as an ACE inhibitor selected from the group consisting of benazepril, captopril, enalapril, fosinopril, lisinopril, moexipril, perindopril, quinapril, ramipril, and trandolapril; an ARB selected from the group consisting of candesartan, eprosartan, irbesartan, telmisartan, valsartan, losartan, and olmesartan; or a combination thereof.

This invention also provides pharmaceutical compositions comprising a compound or salt of the present invention with one or more pharmaceutically acceptable carriers. In a particular embodiment, the pharmaceutical composition further comprises one or more other therapeutic agents, for example, an ACE inhibitor selected from the group consisting of benazepril, captopril, enalapril, fosinopril, lisinopril, moexipril, perindopril, quinapril, ramipril, and trandolapril; an ARB selected from the group consisting of candesartan, eprosartan, irbesartan, telmisartan, valsartan, losartan, and olmesartan; or a combination thereof.

This invention also provides a compound or salt of the present invention for use in therapy. The invention also provides a compound or salt of the present invention for use in the treatment of a kidney disorder (such as CKD or diabetic nephropathy) and/or a diabetic complication. Additionally, this invention provides use of a compound or salt of the present invention in the manufacture of a medicament for treating a kidney disorder (such as CKD or diabetic nephropathy) and/or a diabetic complication. In an embodiment of the invention, the compound or salt of the present invention is for use in simultaneous, separate, or sequential use of an ACE inhibitor selected from the group consisting of benazepril, captopril, enalapril, fosinopril, lisinopril, moexipril, perindopril, quinapril, ramipril, and trandolapril; an ARB selected from the group consisting of candesartan, eprosartan, irbesartan, telmisartan, valsartan, losartan, and olmesartan; or a combination thereof.

In an embodiment of the invention, provided are methods of decreasing proteinuria in a patient, comprising administering to a patient in need of such treatment an effective amount of a compound or salt thereof of the present invention. In an embodiment of the invention, provided is a compound or salt thereof of the present invention for use in decreasing proteinuria. In an embodiment of the invention, provided is the use of a compound or salt thereof of the present invention for the manufacture of a medicament for decreasing proteinuria.

In an embodiment of the invention, provided are methods of decreasing albuminuria in a patient, comprising administering to a patient in need of such treatment an effective amount of a compound or salt thereof of the present invention. In an embodiment of the invention, provided is a compound or salt thereof of the present invention for use in decreasing albuminuria. In an embodiment of the invention, provided is the use of a compound or salt thereof of the present invention for the manufacture of a medicament for decreasing albuminuria.

In an embodiment of the invention, provided are methods of slowing the rate of progression to ESRD in a patient comprising administering to a patient in need of such treatment an effective amount of a compound or salt thereof of the present invention. In an embodiment of the invention, provided is a compound or salt thereof of the present invention, for use in slowing the rate of progression to ESRD in a patient. In an embodiment of the invention, provided is the use of a compound or salt thereof of the present invention, for the manufacture of a medicament for slowing the rate of progression to ESRD.

The term “kidney disorder” means any renal disorder, renal disease, or kidney disease where there is any alteration in normal physiology and function of the kidney. This can result from a wide range of acute and chronic conditions and events, including physical, chemical or biological injury, insult, trauma or disease, such as for example hypertension, diabetes, congestive heart failure, lupus, sickle cell anemia and various inflammatory, infectious and autoimmune diseases, HIV (or related diseases)-associated nephropathies etc. This term includes but is not limited to diseases and conditions such as kidney transplant, nephropathy; chronic kidney disease (CKD); glomerulonephritis; inherited diseases such as polycystic kidney disease; nephromegaly (extreme hypertrophy of one or both kidneys); nephrotic syndrome; end stage renal disease (ESRD); acute and chronic renal failure; interstitial disease; nephritis; sclerosis, an induration or hardening of tissues and/or vessels resulting from causes that include, for example, inflammation due to disease or injury; renal fibrosis and scarring; renal-associated proliferative disorders; and other primary or secondary nephrogenic conditions. Fibrosis associated with dialysis following kidney failure and catheter placement, e.g., peritoneal and vascular access fibrosis, is also included.

In some embodiments, the kidney disorder may be generally defined as a “nephropathy” or “nephropathies”. The terms “nephropathy” or “nephropathies” encompass all clinical-pathological changes in the kidney which may result in kidney fibrosis and/or glomerular diseases (e.g. glomerulosclerosis, glomerulonephritis) and/or chronic renal insufficiency, and can cause end stage renal disease and/or renal failure. In some embodiments, the terms “nephropathy” or “nephropathies” refers specifically to a disorder or disease where there is either the presence of proteins (i.e. proteinuria) in the urine of a subject and/or the presence of renal insufficiency.

The term “fibrosis” refers to abnormal processing of fibrous tissue, or fibroid or fibrous degeneration. Fibrosis can result from various injuries or diseases, and can often result from chronic transplant rejection relating to the transplantation of various organs. Fibrosis typically involves the abnormal production, accumulation, or deposition of extracellular matrix components, including overproduction and increased deposition of, for example, collagen and fibronectin. As used herein, the terms “kidney fibrosis” or “renal fibrosis” or “fibrosis of the kidney” refer to diseases or disorders associated with the overproduction or abnormal deposition of extracellular matrix components, particularly collagen, leading to the degradation or impairment of kidney function.

A “diabetic complication” includes, but is not limited to atherosclerosis, cardiomyopathy, retinopathy, nephropathy, and neuropathy.

The term “patient” includes living organisms in which a kidney disorder (such as chronic kidney disease or diabetic nephropathy), and/or a diabetic complication can occur, or which are susceptible to such pathologies. The term includes animals, (e.g., mammals, e.g., cats, dogs, horses, pigs, cows, goats, sheep, rodents, e.g., mice or rats, rabbits, squirrels, bears, primates (e.g., chimpanzees, monkeys, gorillas, and humans)), as well as chickens, ducks, Peking ducks, geese, and transgenic species thereof. Preferably, the patient is a mammal. More preferably, the patient is a human or a feline.

The terms “treatment,” “treat,” “treating,” and the like, are meant to include slowing or reversing the progression of a disorder. These terms also include alleviating, ameliorating, attenuating, eliminating, or reducing one or more symptoms of a disorder or condition, even if the disorder or condition is not actually eliminated and even if progression of the disorder or condition is not itself slowed or reversed.

“Pharmaceutically acceptable” as used in this application, for example with reference to salts and formulation components such as carriers, includes “veterinarily acceptable”, and thus includes both human and non-human animal applications independently.

The compounds and salts of the present invention are preferably formulated as pharmaceutical compositions, which include veterinary compositions. The pharmaceutical compositions may be administered by a variety of routes. Most preferably, such compositions are for oral or intravenous administration, and include tablets, capsules, solutions, and suspensions. “Carrier” is used herein to describe any ingredient other than the active component(s) in a formulation. The choice of carrier will to a large extent depend on factors such as the particular mode of administration or application, the effect of the carrier on solubility and stability, and the nature of the dosage form. Such pharmaceutical compositions and processes for preparing the same are well known in the art. See, e.g., REMINGTON: THE SCIENCE AND PRACTICE OF PHARMACY (D. Troy, et al., eds., 21^{st} ed., Lippincott Williams & Wilkins, 2005).

The compounds of the present invention are generally effective over a wide dosage range. “Effective amount” means the amount of the compound for the methods and uses of the present invention that will elicit the biological or medical response of, or desired therapeutic effect on, a tissue, system, or patient that is being sought by the researcher, medical doctor, veterinarian, or other clinician. An effective amount of the compound may vary according to factors such as the specific disease involved, the disease state, age, sex, and weight of the patient, the ability of the compound to elicit a desired response in the patient, the response of the patient, the particular compound administered, the mode of administration, the bioavailability characteristics of the preparation administered, the dose regimen selected, and the use of any concomitant medications. An effective amount is also one in which any toxic or detrimental effect of the compound is outweighed by the therapeutically beneficial effects. The frequency of the administration will also be dependent upon several factors, and can be a single or multiple dose administration.

It will be understood by the skilled reader that the compounds of the present invention may capable of forming salts, including pharmaceutically acceptable salts. For example, the compound of Example 1 contains basic amines, and accordingly reacts with any of a number of inorganic and organic acids to form pharmaceutically acceptable acid addition salts. Such pharmaceutically acceptable salts and common methodology for preparing them are well known in the art. See, e.g., P. Stahl, et al., HANDBOOK OF PHARMACEUTICAL SALTS: PROPERTIES, SELECTION AND USE, (VCHA/Wiley-VCH, 2008); S. M. Berge, et al., “Pharmaceutical Salts”, Journal of Pharmaceutical Sciences, Vol. 66, No. 1, January 1977.

The skilled artisan will appreciate that compounds of the present invention may contain at least one chiral center. The present invention contemplates all individual enantiomers or diastereomers, as well as mixtures of the enantiomers and diastereomers of said compounds including racemates. It is preferred that compounds of the present invention containing at least one chiral center exist as single enantiomers or diastereomers. The single enantiomers or diastereomers may be prepared beginning with chiral reagents or by stereoselective or stereospecific synthetic techniques. Alternatively, the single enantiomers or diastereomers may be isolated from mixtures by standard chiral chromatographic or crystallization techniques. For Examples 10 and 31 below, Isomer 1 elutes from the column first and isomer 2 elutes second.

The following Preparations and Examples further illustrate the invention and represent typical synthesis of the compounds of the invention. The reagents and starting materials are readily available or may be readily synthesized by one of ordinary skill in the art. It should be understood that the Preparations and Examples are set forth by way of illustration and not limitation, and that various modifications may be made by one of ordinary skill in the art. The compounds illustrated herein are named using IUPACNAME ACDLABS.

As used herein, the following terms have the meanings indicated: “ATCC” refers to American Type Culture Collection; “ATP” refers to adenosine-5′-triphosphate; “BSA” refers to bovine serum albumin; “DMF” refers to N,N-dimethylformamide; ‘DMSO” refers to dimethyl sulfoxide; “EDTA” refers to ethylenediaminetetraacetic acid; “EGTA” refers to ethylene glycol tetraacetic acid; “h” refers to hour or hours; “HATU” refers to O-(7-azabenzotriazole-1-yl)-N,N,N′,N′-tetramethyluronium hexafluorophosphate; “HTRF” refers to homogeneous time resolved fluorescence; “ICR” refers to imprinting control region; “IgG” refers to immunoglobulin G; “min” refers to minute or minutes; “OGTT” refers to oral glucose tolerance test; “PBS” refers to phosphate buffered saline; “PBMC” refers to peripheral blood mononuclear cells; “Pd(dppf)Cl₂” refers to 1,1′-Bis(diphenylphosphino)ferrocene-palladium(II)dichloride dichloromethane complex; “PKC” refers to protein kinase C; “PMA” refers to phorbol myristate acetate; “P-pleckstrin” refers to phosphorylated pleckstrin; “RPMI” refers to Roswell Park Memorial Institute; “SDS-PAGE” refers to sodium dodecyl sulfate polyacrylamide gel electrophoresis; “STK” refers to serine/threonine kinase; and “THF” refers to tetrahydrofuran.

### Preparation 1

### 5-Bromo-2-fluoro-3-methyl-benzaldehyde

A solution of 4-bromo-1-fluoro-2-methyl-benzene (300 g, 1.59 mol) in THF (1.4 L) is cooled to −65 to −70° C. under nitrogen. Then 2.0 M lithium diisopropylamide in THF (1 L, 2.0 mol, 1.26 eq) is added over 2.5 h, keeping the internal temperature between −65 to −70° C. The reaction temperature is maintained for an additional 1 h after the addition is complete. A solution of DMF (240 mL) and THF (100 mL) are added. The mixture is stirred at −78° C. for 2 h, then warmed to −10° C. over 16 h. Saturated ammonium chloride (5 L) is added, and the mixture is extracted with ethyl acetate (2×3 L). The combined extracts are washed with water (5 L), washed with saturated aqueous sodium chloride (brine) (5 L), and concentrated to give the crude title compound (300 g), which is used without further purification.

### Preparation 2

### 5-Bromo-2-tert-butylsulfanyl-3-methyl-benzaldehyde

To a solution of 5-bromo-2-fluoro-3-methyl-benzaldehyde (480 g, 2.534 mol) in DMF (2.5 L), is added potassium carbonate (600 g, 4.3 mol) followed by 2-methylpropane-2-thiol (300 mL, 2.7 mol). The resulting mixture is heated to 60° C. for 16 h. Water (1.5 L) is added at this temperature, and then the mixture is stirred at 60° C. for an additional 8 h. The reaction mixture is cooled to about 25° C., poured into water (10 L), and then extracted with ethyl acetate (7 L). The organic layer is washed with water (7 L), 5% brine (5 L) and concentrated to afford the crude title compound (600 g), which is used without further purification. LC-ES/MS m/z (⁷⁹Br/⁸¹Br) 230.9/232.9 [M-isobutene+H]⁺.

### Preparation 3

### 5-Bromo-2-tent-butylsulfanyl-3-methyl-benzaldehyde oxime

To a solution of 5-bromo-2-tent-butylsulfanyl-3-methyl-benzaldehyde (600 g, 1.985 mol) in 95% ethanol (3 L) is added hydroxylamine hydrochloride (218 g, 3.1 mol). Then sodium bicarbonate (280 g, 3.34 mol) is added in portions over 10 min After the addition is complete, the reaction mixture is stirred at room temperature for 3 h. The reaction mixture is diluted with water (3 L) and extracted with ethyl acetate (3 L). The organic layer is washed with 5% brine and concentrated. The resulting residue is triturated in petroleum ether (5 L), filtered, and washed with petroleum ether (2×1 L). The cake is air-dried to afford the title compound as a yellow solid (460 g, 63% yield for three steps). LC-ES/MS m/z (⁷⁹Br/⁸¹Br) 304.0/306.0 [M+H]⁺.

### Preparation 4

### 5-Bromo-7-methyl-1,2-benzothiazole

To a suspension of 5-bromo-2-tent-butylsulfanyl-3-methyl-benzaldehyde oxime (460 g, 1.5 mol) in toluene (3 L) is added 4-methylbenzenesulfonic acid (50 g, 0.26 mol). The reaction mixture is heated at 80° C. for 2 h, and then is refluxed for 2 h. The reaction is cooled and diluted with ethyl acetate (3 L). The organic portion is washed with water (3 L), saturated sodium bicarbonate (3 L) and brine (3 L). The organic portion is concentrated. The resulting residue is slurred in petroleum ether (3 L) and filtered. The filter cake is washed with petroleum ether (2×1 L) to afford the title compound as a brown solid (250 g, 72%). LC-ES/MS m/z (⁷⁹Br/⁸¹Br) 227.9/229.9 [M+H]⁺. ¹H NMR (400 MHz, DMSO-d₆) δ 9.08 (s, 1H), 8.26 (s, 1H), 7.57 (s, 1H), 2.54 (s, 3H).

### Preparation 5

### 5-Bromo-1,2-benzothiazole-7-carboxylic acid

To a solution of 5-bromo-7-methyl-1,2-benzothiazole (50 g, 0.22 mol) in carbon tetrachloride (1.5 L) is added N-bromosuccinimide (234.1 g, 1.3 mol) and benzoyl peroxide (10.6 g, 44 mmol). The mixture is heated at reflux for 16 h, cooled to 25° C. and filtered. The filter cake is dissolved in water (2.5 L) and ethyl acetate (2 L). The organic layer and filtrate are combined together and concentrated to give a solid. The residual solid is dried in the air to afford the crude mixture of 5-bromo-7-(dibromomethyl)-1,2-benzothiazole (38% purity detected by UV absorption at 214 nm, LC-ES/MS m/z (⁷⁹Br/⁸¹Br) 384/386/388/390 [M+H]⁺) and 5-bromo-7-(tribromomethyl)-1,2-benzothiazole (28% purity detected by UV absorption at 214 nm, LC-ES/MS m/z (⁷⁹Br/⁸¹Br) 462/464/466/468/470 [M+H]⁺) as a solid (80 g). The crude mixture (80 g) is added to a solution of lithium hydroxide (17.4 g, 0.415 mol) in water (800 mL) and dioxane (800 mL) and heated at reflux for 16 h. The solution is cooled to 25° C. and acidified to pH=2 to 3 with 2 N HCl. The mixture is extracted with ethyl acetate (3×500 mL) and the combined organic portions are concentrated to give a crude mixture of 5-bromo-1,2-benzothiazole-7-carbaldehyde (50% purity detected by UV absorption at 214 nm, LC-ES/MS m/z (⁷⁹Br/⁸¹Br) 242/244 [M+H]⁺) and 5-bromo-1,2-benzothiazole-7-carboxylic acid (8% purity detected by UV absorption at 214 nm, LC-ES/MS m/z (⁷⁹Br/⁸¹Br) 258/260 [M+H]⁺) (100 g). The crude mixture (100 g) is taken up in THF (960 mL), tent-butyl alcohol (320 mL), and water (320 mL). Sodium chlorite (32.2 g, 0.311 mol), sodium dihydrogen phosphate monohydrate (98.3 g, 0.62 mol), and sulfamic acid (32.2 g, 0.33 mol) are added. The mixture is stirred at 25° C. for 16 h, and then concentrated. The resulting residue is purified by triturating with dichloromethane and water (1:1, 400 mL). The slurry is filtered and the cake is dried in air to give the title compound as a solid (26 g, 46% 3-step yield). LC-ES/MS m/z (⁷⁹Br/⁸¹Br) 258/260 [M+H]⁺. ¹H NMR (400 MHz, DMSO-d₆) δ 9.18 (d, J=7.2 Hz, 1H), 8.28 (d, J=2.4 Hz, 1H), 7.12 (d, J=7.6 Hz, 1H), 6.714-6.719 (m, 1H).

### Preparation 6

### (5-Bromo-1,2-benzothiazol-7-yl)-[cis-3,5-dimethylpiperazin-1-yl]methanone

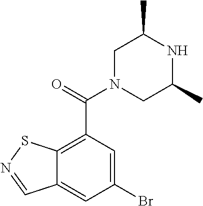 [SMILES: C[C@@H]1CN(C(=O)c2cc(Br)cc3cnsc23)C[C@H](C)N1]

To a mixture of 5-bromo-1,2-benzothiazole-7-carboxylic acid (10 g, 0.039 mol) and DMF (100 mL) is added cis-2,6-dimethylpiperazine (6.64 g, 0.058 mol) at 28° C. The resulting mixture is cooled to 0° C. and HATU (22.1 g, 0.058 mol) is added in portions (internal temperature is 0 to 2° C.). The mixture is warmed to 25° C. and stirred at this temperature for 16 h. The mixture is concentrated under vacuum, poured into water (30 mL) and extracted with ethyl acetate (3×50 mL). The combined organic portions are washed with saturated sodium bicarbonate solution (30 mL) and brine (30 mL). The organic portion is concentrated and the resulting residue is dissolved in a mixture of dichloromethane (30 mL) and water (30 mL). 6 N HCl is added dropwise until a majority of the solid appears. The solid is collected by filtration. The aqueous phase of the filtrate is separated and combined with the solid cake, basified with sodium bicarbonate solution to pH 8, and then extracted with dichloromethane (3×50 mL). The organic phase is concentrated to give the title compound (10.1 g, 74%). LC-ES/MS m/z (⁷⁹Br/⁸¹Br) 354/356 [M+H]⁺.

Preparation 7

### [5-(2,6-Difluoro-4-methoxy-phenyl)-1,2-benzothiazol-7-yl]-[cis-3,5-dimethylpiperazin-1-yl]methanone

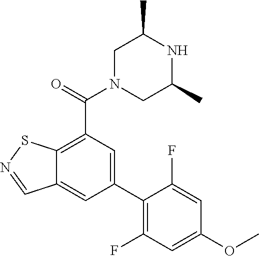 [SMILES: COc1cc(F)c(-c2cc(C(=O)N3C[C@@H](C)N[C@@H](C)C3)c3sncc3c2)c(F)c1]

A mixture of (5-bromo-1,2-benzothiazol-7-yl)-[cis-3,5-dimethylpiperazin-1-yl]methanone (2.0 g, 5.65 mmol), 2,6-difluoro-4-methoxyphenyl boronic acid (1.6 g, 2.47 mmol), [1,1′-bis(diphenylphosphino)ferrocene]dichloropalladium (II) (Pd(dppf)Cl₂) (415 mg, 0.57 mmol), sodium carbonate (1.83 g, 169 mmol), 1,4-dioxane (18 mL), and water (2 mL) is stirred at 85° C. under a nitrogen atmosphere for 16 h. The mixture is cooled to 25° C. and filtered. The filtrate is concentrated under vacuum. The residue is purified by column chromatography on silica gel (eluting with dichloromethane in methanol=150:1 to 100:1). The resulting crude product is dissolved in dichloromethane (20 mL), 4 N HCl in 1,4-dioxane (5 mL, 20 mmol) and a little water is added dropwise. The resulting solid is filtered off and washed with dichloromethane (2×50 mL). The solid is then suspended in water (10 mL), basified with saturated sodium carbonate solution, and extracted with dichloromethane (3×100 mL). The combined organic portions are dried over sodium sulfate, filtered, and the filtrate concentrated to give the title compound (1.1 g, 47%). LC-ES/MS m/z 418.1 [M+H]⁺.

### EXAMPLE 1

### 4-(7-{[(3R,5S)-3,5-DIMETHYLPIPERAZIN-1-YL]CARBONYL}-1,2-BENZOTHIAZOL-5-YL)-3,5-DIFLUOROPHENOL

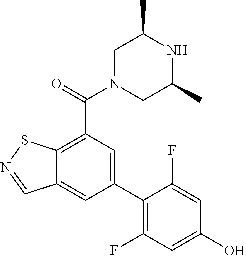 [SMILES: C[C@@H]1CN(C(=O)c2cc(-c3c(F)cc(O)cc3F)cc3cnsc23)C[C@H](C)N1]

To a mixture of 5-(2,6-difluoro-4-methoxy-phenyl)-1,2-benzothiazol-7-yl]-[cis-3,5-dimethylpiperazin-1-yl]methanone (1.0 g, 2.40 mmol) and dichloromethane (30 mL) is added boron tribromide (2.7 mL, 10.54 mmol) dropwise at −78° C. under a nitrogen atmosphere. The mixture is allowed to warm to 25° C. and stirred for 21 h. The reaction is quenched with methanol at −40° C. and basified with ammonia to pH=8. The mixture is extracted with a solution of dichloromethane and isopropyl alcohol (3×160 mL, 3/1 v/v). The combined organic portions are dried over sodium sulfate, filtered, and concentrated. The resulting residue is slurried with methanol (5 mL) and filtered. The filter cake is washed with dichloromethane to give the title compound (0.5 g, 51%). LC-ES/MS m/z 404.0 [M+H]⁺.

### EXAMPLE 2

### 4-(7-{[(3R,5 S)-3,5-DIMETHYLPIPERAZIN-1-YL]CARBONYL}-1,2-BENZOTHIAZOL-5-YL)-3,5-DIFLUOROPHENOL HYDROCHLORIDE

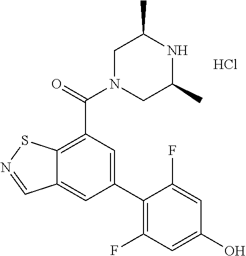 [SMILES: C[C@@H]1CN(C(=O)c2cc(-c3c(F)cc(O)cc3F)cc3cnsc23)C[C@H](C)N1 ; Cl]

To a mixture of [5-(2,6-difluoro-4-hydroxy-phenyl)-1,2-benzothiazol-7-yl]-[cis-3,5-dimethylpiperazin-1-yl]methanone (9.0 g, 22.3 mmol) in methanol (300 mL) and dichloromethane (300 mL) is added 4 N HCl in 1,4-dioxane (30 mL, 120 mmol). The mixture is stirred at 25° C. for 16 h. The reaction mixture is concentrated under vacuum and the residue is washed with methanol (3×50 mL) to give the title compound as a white solid (8.66 g, 88%). LC-ES/MS m/z 404.0 [M+H]⁺. ¹H NMR (300 MHz, DMSO-d₆) δ 10.75(s, 1H), 9.72 (d, J=11.7 Hz, 1H), 9.22 (s, 2H), 8.41 (s, 1H), 7.92 (s, 1H), 6.71 (d, J=10.2 Hz, 2H), 4.32-4.25 (m, 2H), 3.46-3.55 (m, 3H), 3.13-3.17 (m, 1H), 1.27 (d, J=6.3 Hz, 6H).

### EXAMPLE 3

### [5-(2,6-DIFLUORO-4-HYDROXYPHENYL)-1,2-BENZOTHIAZOL-7-YL][(3R,5S)-3,5-DIMETHYLPIPERAZIN-1-YL]METHANONE METHANESULFONATE

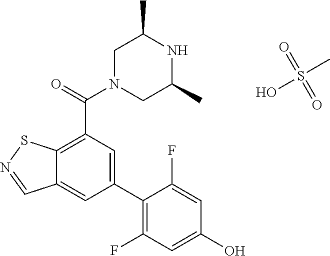

To a solution of [5-(2,6-difluoro-4-hydroxy-phenyl)-1,2-benzothiazol-7-yl]-[cis-3,5-dimethylpiperazin-1-yl]methanone (967.2 mg, 2.4 mmol) in dichloromethane (8 mL) and methanol (30 mL) is added 0.2 N methanesulfonic acid aqueous solution (12 mL, 2.4 mmol). The mixture is stirred at 25° C. for 30 min and concentrated under reduced pressure at 40° C. to give the title compound as a light yellow solid (1.18 g, 98%). ES/MS m/z 404.0 [M+H]⁺. ¹H NMR (300 MHz, CD₃OD) δ 9.13(s, 1H), 8.45 (s, 1H), 7.92 (s, 1H), 6.65 (d, J=13.5 Hz, 2H), 4.55-4.60 (m, 2H), 3.55-3.62 (m, 2H), 3.14-3.34 (m, 2H), 2.75 (s, 3H), 1.42(d, J=6.9 Hz, 6H).

### EXAMPLE 4

### ETHANE-1,2-DISULFONIC ACID-[5-(2,6-DIFLUORO-4-HYDROXYPHENYL)-1,2-BENZOTHIAZOL-7-YL][(3R,5S)-3,5-DIMETHYLPIPERAZIN-1-YL] METHANONE

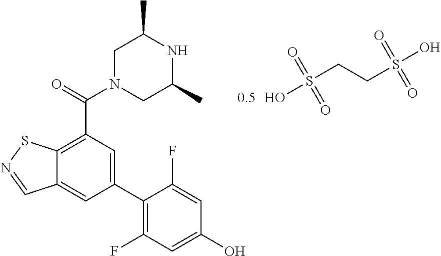 [SMILES: C[C@@H]1CN(C(=O)c2cc(-c3c(F)cc(O)cc3F)cc3cnsc23)C[C@H](C)N1 ; O=S(=O)(O)CCS(=O)(=O)O]

To a solution of [5-(2,6-difluoro-4-hydroxy-phenyl)-1,2-benzothiazol-7-yl]-[cis-3,5-dimethylpiperazin-1-yl]methanone (967.2 mg, 2.4 mmol) in dichloromethane (8 mL) and methanol (30 mL) is added 0.05 N ethane-1,2-disulfonic acid aqueous solution (24 mL, 1.2 mmol). The mixture is stirred at 25° C. for 30 min and concentrated under reduced pressure at 40° C. to give the title compound as a white solid (1.18 g, 99%). LC-ES/MS m/z 404.0 [M+H]⁺. ¹H NMR (300 MHz, DMSO-d₆) δ 10.71 (s, 1H), 9.31 (s, 2H), 8.65 (s, 1H), 7.92 (s, 1H), 6.73 (d, J=16.5 Hz, 2H), 4.33-4.37 (m, 2H), 3.36-3.48 (m, 2H), 3.02-3.14 (m, 2H), 2.50-2.52 (m, 2H), 1.42(d, J=6.3 Hz, 6H).

The following compounds may be prepared in an analagous fashion as set forth in Preparations 1-7 and Examples 1-4.

TABLE 1

| Example | Name | Physical Data |
|---|---|---|
| 5 | [5-(3-FLUORO-4-HYDROXY-2- | LC-ES/MS m/z 428.2 [M + H]+ |
| | METHYLPHENYL)-1,2- | |
| | BENZOTHIAZOL-7-YL](2,2,5,5- | |
| | TETRAMETHYLPIPERAZIN-1- | |
| | YL)METHANONE | |
| | HYDROCHLORIDE | |
| 6 | [5-(2,6-DIFLUORO-4- | LC-ES/MS m/z 418.1 [M + H]+ |
| | HYDROXYPHENYL)-1,2- | |
| | BENZOTHIAZOL-7-YL][(2R,3S,5R)- | |
| | 2,3,5-TRIMETHYLPIPERAZIN-1- | |
| | YL]METHANONE | |
| | HYDROCHLORIDE | |
| 7 | 3-CHLORO-4-(7-{[(3R,5S)-3,5- | LC-ES/MS m/z 419.9 [M + H]+ |
| | DIMETHYLPIPERAZIN-1- | |
| | YL]CARBONYL}-1,2- | |
| | BENZOTHIAZOL-5-YL)-5- | |
| | FLUOROPHENOL | |
| | HYDROCHLORIDE | |
| 8 | ETHANESULFONIC ACID-[5-(2,6- | LC-ES/MS m/z 404.1 [M + H]+ |
| | DIFLUORO-4-HYDROXYPHENYL)- | |
| | 1,2-BENZOTHIAZOL-7-YL][(3R,5S)- | |
| | 3,5-DIMETHYLPIPERAZIN-1- | |
| | YL]METHANONE | |
| 9 | 4-(7-{[(2S,5R)-2,5- | LC-ES/MS m/z 404.2 [M + H]+ |
| | DIMETHYLPIPERAZIN-1- | |
| | YL]CARBONYL}-1,2- | |
| | BENZOTHIAZOL-5-YL)-3,5- | |
| | DIFLUOROPHENOL | |
| | TRIFLUOROACETATE (SALT) | |
| 10 | [5-(2,6-DIFLUORO-4- | LC-ES/MS m/z 432.2 [M + H]+ |
| | HYDROXYPHENYL)-1,2- | |
| | BENZOTHIAZOL-7-YL](2,3,5,6- | |
| | TETRAMETHYLPIPERAZIN-1- | |
| | YL)METHANONE | |
| | HYDROCHLORIDE (Isomer 1) | |
| 11 | 5-CHLORO-4-(7-{[(3R,5S)-3,5- | LC-ES/MS m/z 420.0 [M + H]+ |
| | DIMETHYLPIPERAZIN-1- | |
| | YL]CARBONYL}-1,2- | |
| | BENZOTHIAZOL-5-YL)-2- | |
| | FLUOROPHENOL | |
| | HYDROCHLORIDE | |
| 12 | 4-(7-{[(3R,5S)-3,5- | LC-ES/MS m/z 404.2 [M + H]+ |
| | DIMETHYLPIPERAZIN-1- | |
| | YL]CARBONYL}-1,2- | |
| | BENZOTHIAZOL-5-YL)-3,5- | |
| | DIFLUOROPHENOL | |
| | TRIFLUOROACETATE (SALT) | |
| 13 | [5-(2,6-DIFLUORO-4- | LC-ES/MS m/z 418.1 [M + H]+ |
| | HYDROXYPHENYL)-1,2- | |
| | BENZOTHIAZOL-7-YL][(2R,3S,5R)- | |
| | 2,3,5-TRIMETHYLPIPERAZIN-1- | |
| | YL]METHANONE | |
| | HYDROCHLORIDE | |
| 14 | [5-(2,6-DIFLUORO-4- | LC-ES/MS m/z 404.1 [M + H]+ |
| | HYDROXYPHENYL)-1,2- | |
| | BENZOTHIAZOL-7-YL][(2R,5R)- | |
| | 2,5-DIMETHYLPIPERAZIN-1- | |
| | YL]METHANONE | |
| | HYDROCHLORIDE | |
| 15 | [5-(2,6-DIFLUORO-4- | LC-ES/MS m/z 432.1 [M + H]+ |
| | HYDROXYPHENYL)-1,2- | |
| | BENZOTHIAZOL-7- | |
| | YL][(2R,3S,5R,6S)-2,3,5,6- | |
| | TETRAMETHYLPIPERAZIN-1- | |
| | YL]METHANONE | |
| | HYDROCHLORIDE | |
| 16 | [5-(2,6-DIFLUORO-4- | LC-ES/MS m/z 418.1 [M + H]+ |
| | HYDROXYPHENYL)-1,2- | |
| | BENZOTHIAZOL-7-YL][(5S)-3,3,5- | |
| | TRIMETHYLPIPERAZIN-1- | |
| | YL]METHANONE | |
| | HYDROCHLORIDE | |
| 17 | 3-CHLORO-5-FLUORO-4-(7-{[(3R)- | LC-ES/MS m/z 405.9 [M + H]+ |
| | 3-METHYLPIPERAZIN-1- | |
| | YL]CARBONYL}-1,2- | |
| | BENZOTHIAZOL-5-YL)PHENOL | |
| | HYDROCHLORIDE | |
| 18 | [5-(2,6-DIFLUORO-4- | LC-ES/MS m/z 418.1 [M + H]+ |
| | HYDROXYPHENYL)-1,2- | |
| | BENZOTHIAZOL-7-YL][(3R,5S)- | |
| | 3,4,5-TRIMETHYLPIPERAZIN-1- | |
| | YL]METHANONE | |
| | HYDROCHLORIDE | |
| 19 | 3-CHLORO-4-(7-{[(3R,5S)-3,5- | LC-ES/MS m/z 402.0 [M + H]+ |
| | DIMETHYLPIPERAZIN-1- | |
| | YL]CARBONYL}-1,2- | |
| | BENZOTHIAZOL-5-YL)PHENOL | |
| | HYDROCHLORIDE | |
| 20 | [5-(2-CHLORO-6-FLUORO-4- | LC-ES/MS m/z 433.9 [M + H]+ |
| | HYDROXYPHENYL)-1,2- | |
| | BENZOTHIAZOL-7-YL][(5S)-3,3,5- | |
| | TRIMETHYLPIPERAZIN-1- | |
| | YL]METHANONE | |
| | HYDROCHLORIDE | |
| 21 | [5-(2-CHLORO-5-FLUORO-4- | LC-ES/MS m/z 448.1 [M + H]+ |
| | HYDROXYPHENYL)-1,2- | |
| | BENZOTHIAZOL-7-YL](2,2,5,5- | |
| | TETRAMETHYLPIPERAZIN-1- | |
| | YL)METHANONE | |
| | HYDROCHLORIDE | |
| 22 | 4-(7-{[(3R,5R)-3,5- | LC-ES/MS m/z 404.1 [M + H]+ |
| | DIMETHYLPIPERAZIN-1- | |
| | YL]CARBONYL}-1,2- | |
| | BENZOTHIAZOL-5-YL)-3,5- | |
| | DIFLUOROPHENOL | |
| | HYDROCHLORIDE | |
| 23 | [(3S,5S)-3,5-DIMETHYLPIPERAZIN- | LC-ES/MS m/z 386.1 [M + H]+ |
| | 1-YL][5-(2-FLUORO-4- | |
| | HYDROXYPHENYL)-1,2- | |
| | BENZOTHIAZOL-7- | |
| | YL]METHANONE | |
| | HYDROCHLORIDE | |
| 24 | 3-CHLORO-4-{7-[(3,3- | LC-ES/MS m/z 420.1 [M + H]+ |
| | DIMETHYLPIPERAZIN-1- | |
| | YL)CARBONYL]-1,2- | |
| | BENZOTHIAZOL-5-YL}-5- | |
| | FLUOROPHENOL | |
| | HYDROCHLORIDE | |
| 25 | 4-{7-[(3,3-DIMETHYLPIPERAZIN-1- | LC-ES/MS m/z 400.0 [M + H]+ |
| | YL)CARBONYL]-1,2- | |
| | BENZOTHIAZOL-5-YL}-3- | |
| | FLUORO-5-METHYLPHENOL | |
| | HYDROCHLORIDE | |
| 26 | 2-(7-{[(2S,5R)-2,5- | LC-ES/MS m/z 411.1 [M + H]+ |
| | DIMETHYLPIPERAZIN-1- | |
| | YL]CARBONYL}-1,2- | |
| | BENZOTHIAZOL-5-YL)-3- | |
| | FLUORO-5- | |
| | HYDROXYBENZONITRILE | |
| | HYDROCHLORIDE | |
| 27 | [5-(2-CHLORO-6-FLUORO-4- | LC-ES/MS m/z 418.1 [M + H]+ |
| | HYDROXYPHENYL)-1,2- | |
| | BENZOTHIAZOL-7-YL](4,7- | |
| | DIAZASPIRO[2.5]OCT-7- | |
| | YL)METHANONE | |
| | HYDROCHLORIDE | |
| 28 | [5-(2,5-DIFLUORO-4- | LC-ES/MS m/z 404.1 [M + H]+ |
| | HYDROXYPHENYL)-1,2- | |
| | BENZOTHIAZOL-7-YL][(3R,5S)-3,5- | |
| | DIMETHYLPIPERAZIN-1- | |
| | YL]METHANONE | |
| | HYDROCHLORIDE | |
| 29 | [5-(5-FLUORO-4-HYDROXY-2- | LC-ES/MS m/z 428.2 [M + H]+ |
| | METHYLPHENYL)-1,2- | |
| | BENZOTHIAZOL-7-YL](2,2,5,5- | |
| | TETRAMETHYLPIPERAZIN-1- | |
| | YL)METHANONE | |
| | HYDROCHLORIDE | |
| 30 | [(3R,5S)-3,5- | LC-ES/MS m/z 400.0 [M + H]+ |
| | DIMETHYLPIPERAZIN-1-YL][5-(5- | |
| | FLUORO-4-HYDROXY-2- | |
| | METHYLPHENYL)-1,2- | |
| | BENZOTHIAZOL-7- | |
| | YL]METHANONE | |
| | HYDROCHLORIDE | |
| 31 | [5-(2,6-DIFLUORO-4- | LC-ES/MS m/z 432.2 [M + H]+ |
| | HYDROXYPHENYL)-1,2- | |
| | BENZOTHIAZOL-7-YL](2,3,5,6- | |
| | TETRAMETHYLPIPERAZIN-1- | |
| | YL)METHANONE | |
| | HYDROCHLORIDE (Isomer 2) | |
| 32 | [5-(2-CHLORO-3-FLUORO-4- | LC-ES/MS m/z 420.1 [M + H]+ |
| | HYDROXYPHENYL)-1,2- | |
| | BENZOTHIAZOL-7-YL](3,3- | |
| | DIMETHYLPIPERAZIN-1- | |
| | YL)METHANONE | |
| | HYDROCHLORIDE | |
| 33 | 3-CHLORO-4-(7-{[(2S,5R)-2,5- | LC-ES/MS m/z 419.9 [M + H]+ |
| | DIMETHYLPIPERAZIN-1- | |
| | YL]CARBONYL}-1,2- | |
| | BENZOTHIAZOL-5-YL)-5- | |
| | FLUOROPHENOL | |
| | HYDROCHLORIDE | |
| 34 | [5-(2,5-DIFLUORO-4- | LC-ES/MS m/z 432.2 [M + H]+ |
| | HYDROXYPHENYL)-1,2- | |
| | BENZOTHIAZOL-7-YL](2,2,5,5- | |
| | TETRAMETHYLPIPERAZIN-1- | |
| | YL)METHANONE | |
| | HYDROCHLORIDE | |
| 35 | 3-CHLORO-4-(7-{[(2S,5R)-2,5- | LC-ES/MS m/z 419.9 [M + H]+ |
| | DIMETHYLPIPERAZIN-1- | |
| | YL]CARBONYL}-1,2- | |
| | BENZOTHIAZOL-5-YL)-2- | |
| | FLUOROPHENOL | |
| | HYDROCHLORIDE | |
| 36 | 4-(7-{[(2R,6S)-2,6- | LC-ES/MS m/z 404.2 [M + H]+ |
| | DIMETHYLPIPERAZIN-1- | |
| | YL]CARBONYL}-1,2- | |
| | BENZOTHIAZOL-5-YL)-3,5- | |
| | DIFLUOROPHENOL | |
| | HYDROCHLORIDE | |
| 37 | [(2S,5R)-2,5- | LC-ES/MS m/z 400.2 [M + H]+ |
| | DIMETHYLPIPERAZIN-1-YL][5-(3- | |
| | FLUORO-4-HYDROXY-2- | |
| | METHYLPHENYL)-1,2- | |
| | BENZOTHIAZOL-7- | |
| | YL]METHANONE | |
| | HYDROCHLORIDE | |
| 38 | [5-(2,6-DIFLUORO-4- | LC-ES/MS m/z 418.1 [M + H]+ |
| | HYDROXYPHENYL)-1,2- | |
| | BENZOTHIAZOL-7-YL][(5R)-3,3,5- | |
| | TRIMETHYLPIPERAZIN-1- | |
| | YL]METHANONE | |
| | HYDROCHLORIDE | |
| 39 | 4-{7-[(3,3-DIMETHYLPIPERAZIN-1- | LC-ES/MS m/z 403.9 [M + H]+ |
| | YL)CARBONYL]-1,2- | |
| | BENZOTHIAZOL-5-YL}-3,5- | |
| | DIFLUOROPHENOL | |
| 40 | 3-FLUORO-5-METHYL-4-(7-{[(3R)- | LC-ES/MS m/z 386.0 [M + H]+ |
| | 3-METHYLPIPERAZIN-1- | |
| | YL]CARBONYL}-1,2- | |
| | BENZOTHIAZOL-5-YL)PHENOL | |
| | HYDROCHLORIDE | |
| 41 | 4-[7-(4,7-DIAZASPIRO[2.5]OCT-7- | LC-ES/MS m/z 402 [M + H]+ |
| | YLCARBONYL)-1,2- | |
| | BENZOTHIAZOL-5-YL]-3,5- | |
| | DIFLUOROPHENOL | |
| | TRIFLUOROACETATE (SALT) | |
| 42 | 4-(7-{[(2R,6S)-2,6- | LC-ES/MS m/z 399.9 [M + H]+ |
| | DIMETHYLPIPERAZIN-1- | |
| | YL]CARBONYL}-1,2- | |
| | BENZOTHIAZOL-5-YL)-3- | |
| | FLUORO-5-METHYLPHENOL | |
| | HYDROCHLORIDE | |

### Biological Assays

Considerable literature evidence suggests that the intrarenal renin-angiotensin system plays an important role in diabetic nephropathy. Since PKC is activated by angiotensin II and initial treatment for diabetic nephropathy is with angiotensin-converting enzyme (ACE) inhibitors, angiotensin II receptor blockers (ARBs), or combinations thereof, it is likely that the combination of a PKC inhibitor with an ACE inhibitor, an ARB, or both may present a more effective treatment option for patients with diabetic nephropathy.

Furthermore, the effects of ruboxistaurin, reported to be a selective PKC-β inhibitor, administered in combination with an ACE inhibitor, ARB, or both have been described in the literature. Tuttle, K. T. et al., “The Effect of Ruboxistaurin on Nephropathy in Type 2 Diabetes”, Diabetes Care, Volume 28, No. 11: 2686-2690 (November 2005). It was reported there that ruboxistaurin had favorable effects on albuminuria and renal function in persons with type 2 diabetes and nephropathy.

Therefore, it is believed that a compound that inhibits multiple PKC isoforms, i.e. conventional (including PKC-β) and/or novel isoforms, particularly when used in combination with an ACE inhibitor, ARB, or both, may provide an effective treatment for diabetic nephropathy.

The prevalence of diabetic nephropathy has placed it at the forefront of attempts to discover new therapies for progressive kidney disease. However, non-diabetic kidney diseases or disorders, principally due to various forms of glomerulopathy, remain a major contributor to the number of patients requiring dialysis and transplants. While the aetiologies of these two major categories of kidney disease are clearly different, they share common clinical manifestations such as hypertension, proteinuria (albumin) and declining glomerular filtration rate (GFR) as well as major histopathological characteristics, including glomerulosclerosis, tubulointerstitial fibrosis and macrophage infiltration. Numerous findings also suggest common pathogenetic mechanisms that link diabetic and non-diabetic kidney disease, such as the renin-angiotensin system (RAS) and the elaboration of profibrotic growth factors. Together, these findings raise the possibility that treatments designed to target the diabetic kidney might also be effective in the non-diabetic setting. Kelley, D. J. et al., “Protein Kinase C-β Inhibition Attenuates the Progression of Nephropathy in Non-Diabetic Kidney Disease”, Nephrol. Dial. Transplant, Vol. 24, 1782-1790 (2009). Therefore, it is believed that a compound of the current invention may provide an effective treatment for kidney disorders, including chronic kidney disease.

The following assays demonstrate that the exemplified compounds of the present invention are potent inhibitors of PKC, demonstrate efficacy by reducing albuminuria, are inhibitors of the conventional and novel isoforms of PKC possibly providing a more effective treatment for diabetic nephropathy by targeting multiple diabetic complications simultaneously, and are preferably not potent inhibitors of Akt.

### Assay 1: PKC Isoforms Based HTRF KINEASE™-STK Jumbo Assay Protocol

The purpose of this assay is to evaluate the inhibitory activity of compounds on various PKC isoforms. It has been reported that perturbations in vascular cell homeostasis caused by different PKC isoforms (PKC-α, β_{1/2}, and PKC-δ) are linked to the development of pathologies affecting large vessel (atherosclerosis, cardiomyopathy) and small vessel (retinopathy, nephropathy, and neuropathy) complications. Geraldes, P. and King, G. L., “Activation of Protein Kinase C Isoforms and Its Impact on Diabetic Complications”, Cir. Res., 106: 1319-1331 (2010). So a compound that inhibits multiple conventional and novel PKC isoforms may provide a more effective treatment option for diabetic nephropathy by targeting multiple diabetic complications simultaneously.

Effects of a test compound on the kinase activity of PKCα, β₂, δ, ε, ι, and ζ, are determined using a HTRF KINEASE™-STK assay kit (Cisbio) in accordance with the protocol provided by the vendor. It is a general method for measuring Serine/Threonine kinase activities using kinase substrates and a universal detection system. In the current protocol, biotinilated STK1provided in the assay kit is used as the substrate that can be phosphorylated by any PKC isoform. The HTRF KINEASE™-STK assay kit format involves the two steps (Enzymatic step and Detection step) described below. First, the kinase phosphorylates the substrate in the presence of ATP. Second, the detection reagents provided in the assay kit recognize the phosphorylated substrate. Detection reagents include the STK-antibody labeled with Eu³⁺-Cryptate and streptavidin-XL665. The antibody recognizes only the phosphorylated form of the STK substrate. Streptavidin binds to biotin attached to the kinase substrate. Cryptate serves as the energy donor, while XL665 fluorophor associated with streptavidin serves as an acceptor. Physical proximity between the donor and acceptor results in the fluorescence resonance energy transfer and associated fluorescent signal. Intensity of the signal is proportional to the level of substrate phosphorylation.

Staurosporin (Sigma) may be used as a positive control. It is prepared in DMSO to make up a 10 mM stock solution and then serially diluted (10 μM-0.005 μM) to obtain a ten-point dilution curve. Test compounds are prepared in a similar manner with the top dose at 100 μM. Each compound is serially diluted to obtain a ten-point dilution curve.

The reaction system that is used in the Enzymatic step, consists of the reaction buffer, substrate, and an enzyme. Two kinds of reaction buffers are prepared from the kinase buffer for different PKC isoforms. 5× Kinase Buffer is provided as a part of the kit and consists of HEPES 250 mM (pH7.0), NaN₃ 0.1%, BSA 0.05%, and Orthovanadate 0.5 mM.

For PKCα and PKCε, 1.25× Reaction Buffer consists of 12.5 mM MgCl₂, 1.25 mM DTT, 0.125 mM CaCl₂, and 1.25× Kinase Buffer. For PKCβ₂, PKCδ, PKCι, and PKCζ, 1.35× Reaction Buffer is comprised of 12.5 mM MgCl₂, 1.25 mM DTT, 1.25 mM lipid activator (Upstate Biotechnology, Inc.), and 1.25× Kinase Buffer. These buffers are used for preparation of the substrate mix and enzyme mix. Substrate and enzyme mix differ for various PKC isoforms. For PKCα, the substrate mix consists of 50 μM ATP and 2.5 μM STK1, while the enzyme mix is represented by 3 nM PKCα. For PKCβ2, the substrate mix consists of 25 μM ATP and 2.5 μM STK1, while the enzyme is represented by 3.75 nM of PKCβ₂. For PKCδ, the substrate mix consists of 62.5 μM ATP and 2.5 μM STK1, while the enzyme is represented by 3.75 nM PKCδ. For PKCε, the substrate mix consists of 25 μM ATP and 2.5 μM STK1, while the enzyme mix is represented by 3 nM PKCε. For PKCι, the substrate mix consists of 87.5 μM ATP and 2.5 μM STK1, while the enzyme is represented by 7.5 nM PKCι. For PKCζ, the substrate mix consists of 10 μM ATP and 2.5 μM STK1, while the enzyme is represented by 7.5 nM PKCζ.

For each PKC isoform, to generate 50 μL of reaction system, 10 μL of diluted compound is transferred from a dilution plate to an assay plate, using a Thermo Scientific Matrix. Then 20 μL of substrate mix is added to the assay plate (using a MULTIDROP® Combi dispenser, ThermoScientific) and centrifuged at 500 rpm for 1 min. Finally, 20 μL of enzyme mix is added to the assay plate (using the MULTIDROP®) to initiate the reaction. The plate is shaken for 60 sec on a platform shaker and centrifuged at 1000 rpm for 1 min. The reaction system is incubated at room temperature for various time periods specific for individual PKC isoforms: 30 min for PKCα, PKCβ₂, and PKCε; 40 min for PKCδ; 70 min for PKCι and PKCζ.

In the Detection step, the detection mix contains 0.5625 μM of STK-antibody labeled with Eu3⁺-Cryptate, 0.1563 uM streptavidin-XL665 and EDTA. After incubation with the reaction system, the enzymatic reaction is stopped by adding 40 μL of detection mix (using the MULTIDROP®). The detection mix is capable of stopping the enzymatic reaction due to the presence of EDTA. Solutes are mixed (using a platform shaker) for 1 min and centrifuged at 1000 rpm for 1 min The plates are incubated at room temperature for 1 h (protected from light), and then the fluorescence is measured at 620 nm (Cryptate) and 665 nm (XL665) using Victor-3. A ratio is calculated (665/620) for each well. Specific signal is calculated as a Ratio (Sample)−Ratio (Negative control). The data are processed using XLFIT ® Software (IDBS).

Following a protocol essentially as described above for human PKCα, β₂, δ, ε, ι, and ζ enzyme inhibitor assays, the results demonstrate that exemplified compounds of the invention exhibit selective inhibition of the desired conventional and novel PKC isoforms α, β₂, δ, ε, as compared to the atypical PKC isoforms ι and ζ. For example, the compound of Example 2 has IC₅₀s of about 16.6 nM, 51.0 nM, 68.0 nM, 21.9 nM, >20,000 nM, and 15,500 nM, respectively. Staurosporin, tested in this assay concurrently with the compound of Example 2, has IC₅₀s of about 30.5 nM, 76.1 nM, 27.9 nM, 0.8 nM, 69.4 nM, and 141.9 nM, respectively for PKCα, β₂, δ, ε, ι, and ζ.

### Assay 2: Cell-Based Assay of PKC Inhibition

The purpose of this assay is to test the inhibitory effects of a test compound on PKC in a cell-based system by measuring the level of PKC substrate phosphorylation.

Staurosporin may be used as a positive control. It is prepared in DMSO to make up a 10 μM stock solution and then serially diluted in plain RPMI 1640 culture medium (10 μM-0.0005 μM) to obtain a ten-point dilution curve. Test compounds are prepared in the same manner.

THP-1 cells (human macrophage cell line) are obtained from ATCC (ATCC TIB-202 lot 4028542). The cells are cultured in 96-well plates coated with poly-d-lysine (Becton Dickinson) with seeding density of 4000 cells/well in RPMI 1640 culture medium (Gibco) with 1% fetal bovine serum (Gibco). Cells are treated with test compound, dosing at 10 points of 1:3 dilutions across the range of 10 μM to 0.0005 μM, and with final DMSO concentration at 0.2% for 1.5 h at 37° C. prior to PKC stimulation. “Stimulation” refers to PKC activation by an exogenous stimulus. PKC is stimulated with 20 nM phorbol-12-myristate 13-acetate (PMA, Sigma, P1585) for 30 min at 37° C.

After the incubation, the cells are first fixed in the Prefer fixative (ANATECH LTD) for 30 min at room temperature. Then the fixative is aspirated. 50 μL/well of 0.1% TRITON® X-100 in PBS without Ca++ and Mg++ is added to permeabilize the cells. The cells are permeabilized for 15 min at room temperature. The plates are washed twice with PBS (100 μL/well), and 50 μL/well of the primary antibody solution is added. The cells are incubated with the primary antibody overnight at 4° C., washed with PBS, and then incubated with the labeled secondary antibody for one hour at room temperature. The primary antibody is an anti-phospho-(Serine) PKC substrate IgG (Cell Signaling), diluted 1:1000 in PBS with 1% BSA. The secondary antibody is ALEXA FLUOR® 488 Goat anti-Rabbit IgG (Molecular Probes) diluted 1:1000 in PBS. The plates are washed with PBS. The nuclei are counterstained with 50 μL of 15 μM propidium iodide solution (Molecular Probes) and 50 μg/mL ribonuclease (Sigma) for one hour at room temperature. Plates are scanned with an ACUMEN EXPLORER™ Laser-scanning fluorescence microplate cytometer (TTP LABTECH LTD), with 488 nm laser excitation to detect emission fluorescence at 655 nm-705 nm (emission of DNA bound propidium iodide) for cell counting/well, and emission of 505 nm-530 nm fluorescence of antibody binding to phosphorylated PKC substrates in cells. The main assay output is a ratio of total fluorescence of phosphorylated PKC substrates to total cell number (ratio=total intensity/total cell number). The ratio is used to calculate the dose response and IC₅₀.

Following a protocol essentially as described above for determining PKC substrate phosphorylation in human THP-1 cells, exemplified compounds of the invention demonstrate PKC inhibitory effects in the cell, with most having an absolute IC50 (μM) of 1 or less. For example, the compound of Example 2 has an IC₅₀ of about 0.80 μM. Staurosporin, tested in this assay has an IC₅₀ of about 0.011 μM (conducted in conjunction with the compound of Example 2).

### Assay 3: PKC In Vivo Target Inhibition Assay

The purpose of this assay is to evaluate the effects of in vivo administration of a test compound on PKC substrate phosphorylation in blood cells.

Pleckstrin is one of the PKC protein substrates and is primarily expressed in platelets and peripheral blood mononuclear cells (PBMC). Pleckstrin phosphorylation is proportional to PKC activity. The phosphorylation state of the pleckstrin protein in purified platelets and PBMC from mouse blood is analyzed via Western blotting using a primary antibody specific for phosphorylated PKC substrates. This antibody recognizes multiple phosphorylated PKC substrates that include, but are not limited to p-pleckstrin. The p-pleckstrin band is identified based on its molecular weight.

Male ICR mice (Charles River) are dosed by oral gavage with a test compound homogenized in 1% hydroxethylcellulose/0.25% TWEEN® 80, at a concentration range of 0.3-3 mg/mL, depending on dose; thus dosing volume is 10 ml/kg body weight. Staurosporine, dissolved in the same vehicle, may be used as a positive control to verify that the assay is functioning. The mice are sacrificed 2 h after treatment by CO₂ asphyxiation, and blood is collected via cardiac puncture. Blood is treated with EDTA anti-coagulant as well as protease and phosphatase inhibitors Protease Inhibitor (Roche, #1836170), Phosphatase Inhibitor I (Sigma, P2850), and Phosphatase Inhibitor II (Sigma, P5726).

Platelets are prepared from mouse blood by low speed spin at 200× g for 4 min. Platelet-rich plasma is removed and transferred to an eppendorf tube which is then spun at 1,400× g for 5 min Platelet pellets are suspended in a small volume of lysis buffer (150 mM NaCl, 20 mM Tris (pH 7.5), 1 mM EDTA, 1 mM EGTA, 1% TRITON X-100®, Protease Inhibitor (Roche, #1836170), Phosphatase Inhibitor I (Sigma, P2850), and Phosphatase Inhibitor II (Sigma, P5726)) and frozen until further analysis. The remaining blood is mixed and applied to BD VACUTAINER® CPT™ tubes (Becton Dickinson) to purify PBMC via centrifugation at 1,600-1,800× g for 20-25 minutes. The PBMC band is recovered with a pipette, diluted with pH 7.5 buffer and spun down at 1,400× g for 5 min, suspended in a small volume of lysis buffer and frozen until further analysis. Proteins are obtained by incubation of the cells with the lysis buffer.

10% SDS-PAGE is conducted on the cell lysates (30 μg protein loaded per lane), with 130 V for 90 min. After transfer, nitrocellulose membranes are incubated with the rabbit polyclonal anti-phospho-PKC substrate antibody (Cell Signaling Technology Inc). The membranes are washed and incubated with the secondary conjugated fluorescent anti-rabbit antibody (ALEXA FLUOR® 680 goat anti-rabbit IgG, Invitrogen Molecular Probes). The membranes are scanned with an ODYSSEY® Infrared Imaging System (LI-COR). The phospho-pleckstrin band in the gel is identified by its relative migration in the gel relative to molecular weight marker proteins, and (in selected cases) by phospho-proteomic analysis. Intensity of near-infrared fluorescence is proportional to the level of PKC substrate phosphorylation. It is quantitated with ODYSSEY® software. For a dose-response experiment, the optical density values (y) are averaged and plotted vs dose values (x) to generate the ID₅₀.

Following a protocol essentially as described above for determining pleckstrin phosphorylation by PKC in vivo, the compound of Example 2 has an ID₅₀ of about 11.85 mg/kg for the platelets and about 18.03 mg/kg for the PBMCs. This result demonstrates that Example 2 has inhibitory effects on PKC substrate phosphorylation in vivo.

### Assay 4: Efficacy Animal Model of Diabetic Nephropathy

The purpose of this assay is to analyze compound efficacy in a mouse model of diabetic nephropathy. The earliest clinical manifestation of diabetic nephropathy is albuminuria, leakage of albumin in the urine. Because diabetic nephropathy can be present without any symptoms, early diagnosis is critical so that treatment can be started. The key test for early diagnosis of diabetic nephropathy involves checking for the presence of albuminuria. Thus, if a compound reduces albumin levels in the urine in a diabetic patient, it would likely indicate efficacy of the compound in the treatment of diabetic nephropathy.

To model diabetic nephropathy, a combination of genetically driven type 2 diabetes and uninephrectomy is utilized. Six week-old db/db mice (genetic strain: C57 BL KsJ, ChemPartner, Shanghai) receive standard rodent chow and water ad libitum. The left kidney is removed under anesthesia with 0.6% sodium pentobarbital at 60 mg/kg body weight (10 μL/g i.p.). Seven days after surgery, urine is collected for 24 h in a metabolic cage to measure albuminuria levels and blood is collected by tail snip to determine glucose levels. For compound treatment, the animals are randomized based on 24-h albuminuria, blood glucose, and body weight. A test compound is dissolved in 4% DMSO (aqueous) and administered twice a day by oral gavage. Control and treatment groups consisted of 10 mice each. The control group is treated with the vehicle (4% DMSO) in a similar manner. During the treatment period, albuminuria is analyzed monthly. After 2 months of treatment, blood is collected by heart puncture under isofluorane anesthesia, and the mice are euthanized by removing the heart.

Following a protocol essentially as described above for determining reduction of albuminuria in db/db mice, the compound of Example 2 has an ED₅₀ of about 25.33 mg/kg. Specifically, at the dose of 30 mg/kg, actual mean levels of albumin in the urine (collected during 24 hours) were 85.0±6.9 μg (n=12) while in the urine of vehicle-treated mice they were 135.9±14.3 μg (n=12). This result demonstrates that the compound of Example 2 reduces albumin levels in the urine of mice in an animal model of diabetic nephropathy.

1. A compound of the formula: 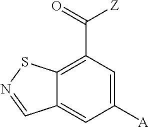
or a salt thereof, wherein:
A is 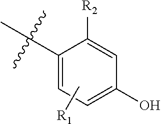
R₁ and R₂ are each independently hydrogen, fluoro, chloro, methyl, or cyano, wherein at least one of R₁ or R₂ is not hydrogen;
Z is 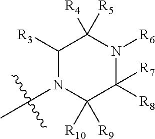
R₃, R₆, R₉, and R₁₀ are each independently hydrogen or methyl;
R₄ and R₅ are each independently hydrogen or methyl, or R₄ and R₅ taken together with the carbon to which they are attached form cyclopropyl; and
R₇ and R₈ are each independently hydrogen or methyl, or R₇ and R₈ taken together with the carbon to which they are attached form cyclopropyl; wherein at least one of R₃, R₄, R₅, R₇, R₈, R₉, or R₁₀ is not hydrogen.

2. The compound or salt according to claim 1, wherein A is

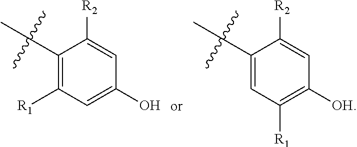

3. The compound or salt according to claim 1, wherein R₂ is fluoro.

4. The compound or salt according to claim 1, wherein A is

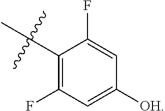 [SMILES: *c1c(F)cc(O)cc1F]

5. The compound or salt according to claim 1, wherein Z is

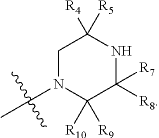

6. The compound or salt according to claim 5, wherein Z is

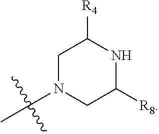

7. The compound or salt according to claim 6, wherein Z is

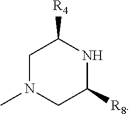

8. A compound or salt according to claim 1, which is:

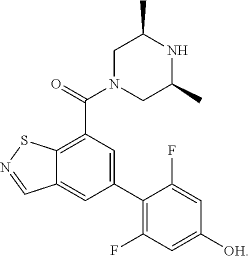 [SMILES: C[C@@H]1CN(C(=O)c2cc(-c3c(F)cc(O)cc3F)cc3cnsc23)C[C@H](C)N1]

9. The hydrochloride salt, the methane sulfonic acid salt, or the hemi ethane-1,2-disulfonic acid salt of the compound according to claim 8.

10. A pharmaceutical composition comprising the compound or salt according to claim 1 and one or more pharmaceutically acceptable carriers.

11. The pharmaceutical composition according to claim 10 further comprising one or more additional therapeutic agents.

12. A method of treating a kidney disorder in a patient in need thereof comprising administering a compound or salt according to claim 1.

13. The method of claim 12, wherein said kidney disorder is chronic kidney disease.

14. The method according to claim 12, wherein the kidney disorder is diabetic nephropathy.

15. The method according to claim 12, wherein said patient is human.

16. The method according to claim 12, wherein said patient is feline.

17. A method of treating a diabetic complication in a patient in need thereof comprising administering a compound or salt according to claim 1.

18. The method of claim 17, wherein said diabetic complication is selected from the group consisting of atherosclerosis, cardiomyopathy, retinopathy, nephropathy, and neuropathy.

19.-24. (canceled)
